(19) United States
(12) Patent Application Publication
(10) Pub. No.: US 2006/0135610 A1
(43) Pub. Date: Jun. 22, 2006

(54) Cardiovascular compositions

(76) Inventors: Jonathan David Bortz, St. Louis, MO (US); Mitchell I. Kirschner, St. Louis, MO (US); Marc S. Hermelin, St. Louis, MO (US); David S. Hermelin, St. Louis, MO (US)

Correspondence Address: BLACKWELL SANDERS PEPER MARTIN LLP, 720 OLIVE STREET, SUITE 2400, ST. LOUIS, MO, 63101, US

(21) Appl. No.: 11/021,282

(22) Filed: Dec. 22, 2004

(51) Int. Cl. A61K 31/202; A61K 31/225

(52) U.S. Cl. 514/548; 514/560

(57) ABSTRACT

Compositions that promote and/or maintain cardiovascular health through the treatment of one or more cardiovascular diseases are provided. Also provided are methods for using compositions that promote and/or maintain cardiovascular health through the prevention, stabilization, reversal and/or treatment of coronary artery disease and/or cerebrovascular disease. Such compositions may be used independently to promote and/or maintain cardiovascular health or used in combination with one or more other compositions used in the treatment of various other disease states common to aging and/or a health deteriorating condition.

### FIELD OF THE INVENTION

The present invention relates to compositions that promote and/or maintain cardiovascular health through the treatment of one or more cardiovascular diseases. More specifically, the present invention relates to compositions that promote and/or maintain cardiovascular health through the prevention, stabilization, reversal and/or treatment of coronary artery disease and/or cerebrovascular disease. Compositions of the present invention may be used independently to promote and/or maintain cardiovascular health or used in combination with one or more other compositions used in the treatment of various other disease states common to aging or a health deteriorating condition.

### BACKGROUND OF INVENTION

Cardiovascular diseases remain a major cause of death in countries throughout the world. It is estimated that 30 million Americans have some form of cardiovascular disease. Of these 30 million people, more than 4 million people have overt clinical signs of atherosclerosis, primarily of the coronary, cerebral and peripheral blood vessels, and over 23 million people have hypertension as defined by a blood pressure of 160/95 or higher. Furthermore, the latter two major etiologic processes, atherosclerosis and hypertension, are interactive and result in an estimated 1.25 million heart attacks and 500,000 strokes a year. In 1975, cardiovascular diseases accounted for 994,513 deaths, 52.5 percent of all deaths in the United States, of which almost 650,000 were due to coronary artery disease (heart attack and sudden death) and about 194,000 were due to cerebrovascular disease (stroke). Since that time, much has been written concerning the role that diet and other factors play in the prevalence of cardiovascular diseases such as coronary artery disease (CAD) and cerebrovascular disease (CD). Cardiovascular diseases, such as CAD and CD are associated with endothelial cell dysfunction. Endothelial cell dysfunction is characterized by an endothelial cell's loss of barrier function. Such cellular dysfunction allows for infiltration of cellular material through the dysfunctional endothelial cells and into subendothelial cell layers that comprise vascular walls. Loss of endothelial cell integrity or barrier function occurs as a result of shear forces, hypertension, immune complexes, viruses, excessive glucose and/or hyperlipidemia denuding endothelial cell surfaces. Vascular endothelial cell dysfunction likewise causes a loss of nitric oxide (NO) mediated physiological vasodilation, an increase in endothelial cell adhesion and a migration of leucocytes, macrophages and lipoproteins into the subendothelial vascular wall.

A link has been established between inflammation and cardiovascular events mediated by inflammation-induced dysfunction of vascular endothelium cells such as for example arterial endothelium cells. Even mild systemic inflammatory responses are associated with significant alterations in endothelial cell function leading to increased cardiovascular health risks. Thrombus formation is the proximate cause of myocardial infarction. However, atherosclerosis is the chief underlying cause of myocardial infarction. Atherosclerosis is a chronic disease that progresses over decades of life. Inflammation plays a role in both the initiation and the progression of atherosclerosis.

Atherosclerosis may be considered as an aberrant form of wound-healing in arteries. Repeated minor trauma, may well account for the tendency of atherosclerosis to occur mostly at major blood vessel flexion sites and at sites of mechanical stress, such as the bifurcation of the carotid artery. Gaps between vascular endothelial cells allow the insinuation of monocytes and macrophages beneath the endothelium, where macrophages may engulf liquid droplets to become foam cells, which lead to the formation of atherosclerotic plaque.

A misconception exists that CAD primarily affects men. While there is a ten year lag between onset and peak incidence of cardiovascular events in women, the annual number of deaths due to CAD is greater in women than in men. Recently, questions have been raised regarding possible cardiovascular-protective benefits provided through hormone replacement therapy in menopausal women. However, in many instances, healthcare providers have abandoned the use of hormone replacement therapy for menopausal women due to other potential health risks, to which hormone replacement therapy may contribute. Accordingly, there is a need to provide menopausal women, especially those with known risk factors for cardiovascular disease, with means to minimize those risks.

It is clear that there are many key factors that can contribute to the initiation and progression of atherosclerosis. Conventional nutritional supplements have typically provided only one ingredient for the promotion of cardiovascular health. For example, physicians conventionally prescribe folic acid for the single purpose of lowering homocysteine blood levels. High blood levels of homocysteine have been associated with an increased risk of CAD and CD. There is therefore a need for a composition that broadly promotes cardiovascular health.

### SUMMARY OF THE INVENTION

The present invention relates to compositions for administration to humans or other animals to promote and/or maintain cardiovascular health through prevention, stabilization, reversal and/or treatment of cardiovascular diseases such as coronary artery disease (CAD) and cerebrovascular disease (CD). The present compositions preferably comprise an effective amount of one or more endothelial cell anti-inflammatory agents, one or more nitric oxide generation promoting agents, one or more antioxidant agents and one or more platelet aggregation lowering agents. Cardiovascular health is promoted and/or maintained though use of the present compositions by reducing the detrimental effects of endothelial cell inflammation, low nitric oxide generation, low antioxidant activity and platelet aggregation.

The present invention likewise provides methods for treating a human or other animal by administering one or more compositions comprising an effective amount of one or more endothelial cell anti-inflammatory agents, one or more nitric oxide generation promoting agents, one or more antioxidant agents, and one or more platelet aggregation lowering agents, to promote and/or maintain cardiovascular health. The practice of this invention involves administering to humans or other animals by oral, intraperitoneal, intravenous, subcutaneous, transcutaneous or intramuscular routes of administration one or more compositions of the present invention.

The present invention likewise provides methods of manufacturing compositions comprising an effective amount of one or more endothelial cell anti-inflammatory agents, one or more nitric oxide generation promoting agents, one or more antioxidant agents and one or more platelet aggregation lowering agents, to promote and/or maintain cardiovascular health.

Accordingly, it is an object of the present invention to provide a composition effective in the prevention, stabilization, reversal and/or treatment of one or more cardiovascular diseases.

Another object of the present invention is to provide a safe composition for the prevention, stabilization, reversal and/or treatment of coronary artery disease and/or cerebrovascular disease.

Another object of the present invention is to provide an effective method of preventing, stabilizing, reversing and/or treating one or more cardiovascular diseases.

Another object of the present invention is to provide a safe method of preventing, stabilizing, reversing and/or treating one or more cardiovascular diseases.

Another object of the present invention is to provide a method of manufacturing a safe composition for the prevention, stabilization, reversal and/or treatment of one or more cardiovascular diseases.

Still another object of the present invention is to provide a method of manufacturing a composition effective in the prevention, stabilization, reversal and/or treatment of one or more cardiovascular diseases.

These and other objectives and advantages of the present invention, some of which are specifically described and others that are not, will become apparent from the detailed description and claims that follow.

### BRIEF DESCRIPTION OF THE DRAWINGS

FIG. 1 is a chart illustrating the metabolic pathways for the desaturation and elongation of Omega-3 and Omega-6 polyunsaturated fatty acids (PUFAs) and eicosanoid production.

### DETAILED DESCRIPTION OF THE INVENTION

Cardiovascular patients can be categorized according to any one of several risk factor evaluation models. Cardiovascular patients may be and often are categorized based on their personal cardiovascular risk factors. Risk factors are particular characteristics found in healthy individuals that have been noted in observational epidemiologic studies to appear related to the subsequent occurrence of a particular disease. Risk factors typically are not the cause of the disease. Risk factors may be modifiable, such as for example eating habits and activity level, as well as non-modifiable, such as for example age, sex, perimenopausal or menopausal status and family history. Coronary artery disease (CAD) has been associated with hundreds of risk factors and is recognized now to have a complex multi-factorial etiology. The presence or absence and the degree of severity of these risk factors has led to the creation of multiple risk factor evaluation models aimed at predicting the chances of developing CAD for purposes of intervening to minimize or prevent the onset or manifestation of CAD. The most widely accepted risk factor evaluation models are those created from large longitudinal epidemiologic studies. Three such risk factor evaluation models are described in the following publications:
Framingham Study, A Statement for Health Professionals, Circulation 1991; 83:356-362;
An Updated Coronary Risk Profile, Anderson, K. M., Wilson, P. W. F., Odell, P. M., et al; the European Coronary Risk Chart, European Heart Journal (1994) 15, 1300-1331, Prevention of Coronary Heart Disease in Clinical Practice, Recommendations of the Task Force of the European Society of Cardiology, European Atherosclerosis Society and European Society of Hypertension, Pyorala, K., De Backer, G., Graham, I., et al.; and
The Cardiovascular Risk Assessment Algorithm, Clinical Chemistry 2001;47(1)28-30, Proposed Cardiovascular Risk Assessment Algorithm Using High-Sensitivity C-Reactive Protein and Lipid Screening, Rifai, N., Ridker, P. M. These three illustrative risk factor evaluation models are well known to those skilled in the art. Risk factor charts developed from particular risk factor evaluation models allow health professionals to assess a cardiovascular patient's risk of cardiovascular disease based on very low, low, moderate, high and very high risk categories. Each of the above noted risk factor evaluation models recognizes common characteristics of age, cigarette smoking, hypertension, lipid abnormalities, family history, diabetes and the like as major determinants of cardiovascular disease risk.

While the multi-factorial cause of cardiovascular disease has been appreciated for decades, many risk factor evaluation models have placed heavy emphasis on assigning risk and hence treatment strategies on the composition of blood lipid profiles. This is the result of fifty years of heavy focus on the hypothesis that a poor lipid profile, i.e., low high density lipoprotein (HDL) and high low density lipoprotein (LDL) cholesterol levels, is the most important predictor of cardiovascular disease, such as CAD. However, lipid profiling or cholesterol screening fails to identify almost 50% of individuals that ultimately experience a cardiac event. It is estimated that as many as one third of all coronary thromboses occur among individuals with none of the traditionally recognized risk factors, such as hypercholesterolemia, hypertension and smoking. Still, modifiable risk factors remain the focus to promote cardiovascular health due to opportunities to reduce or eliminate the risk factors. Modifiable risk factors may be reduced or eliminated through various lifestyle changes to ultimately improve biochemical and physiologic parameters.

Due to the heavy focus on blood lipid profiles and the like, dietary recommendations and nutrient goals have been established to lower blood lipid levels for the prevention of various cardiovascular diseases. Modified dietary recommendations have also been specified based on the presence of specific risk factors such as various degrees of lipid abnormality and the presence or absence of other modifiable or non-modifiable risk factors. Lipid lowering and anti-hypertensive pharmacotherapy recommendations are likewise based in part on an appreciation of various risk factors before, or often after, a cardiovascular event. The challenge lies in screening out remote or inconsequential risk factors in an attempt to identify real modifiable associations that affect morbidity and mortality. However, no matter how strong the relationship between a particular risk factor and cardiovascular disease, if that risk cannot be easily modified, the value of that knowledge from a public health standpoint is exponentially diminished. The greater the ease of implementing a meaningful risk factor reduction or elimination strategy, the more valuable the strategy. Accordingly, compositions effective in promoting and/or maintaining cardiovascular health are particularly beneficial due to ease of implementation.

In accordance with the present invention, compositions are thereby formulated to beneficially affect key factors of general health and/or cardiovascular disease believed to affect initiation and progression of atherosclerosis. Such initiation and progression of atherosclerosis may be as a natural result of aging or as a result of one or more treatments and/or one or more disease states associated with aging and/or a health deteriorating condition. Such key factors include but are not limited to low antioxidant activity, low nitric oxide (NO) generation, smooth muscle proliferation, cellular inflammation, cellular adhesion, platelet aggregation, coagulation and advanced glycated end product (AGEP) formation. Compositions of the present invention are formulated to affect particular key factors of cardiovascular disease. For example, a composition of the present invention may be formulated to effectively decrease cellular inflammation and cellular adhesion, increase NO generation, increase antioxidant activity and decrease platelet aggregation. Likewise, compositions of the present invention may be formulated based on such key factors to meet specific needs of particular classes of cardiovascular patients as established by recognized risk factor evaluation models or cardiovascular risk classifications.

The practice of this invention involves administering to humans or other animals either enterally or parenterally such as by oral, intraperitoneal, intravenous, subcutaneous, transcutaneous or intramuscular routes of administration one or more compositions of the present invention. A dosage of one or more compositions of the present invention may be manufactured in one or more dosage forms such as for example but not limited to a tablet, caplet, capsule, gel capsule, chew tablet, lozenge and troche, nutritional bar or food item, soft chew, reconstitutable powder or shake, sprinkle, semi-solid sachet or the like. Any tablet dosage form may be either chewable or compressed. The preferred solid dosage form for purposes of the present invention is a gel capsule. However, compositions of the present invention could likewise be incorporated into a food product or a powder for mixing with a liquid. Although any number of suitable dosage forms can be used to administer compositions of the present invention, preferred dosage forms include a single gel capsule, two gel capsules or one gel capsule and one caplet or tablet.

Compositions of the present invention can not only be provided in various dosage forms but can also be administered in accordance with various dosage regimens. For example, a dosage of one or more compositions of the present invention may be administered in the form of 1 to 20 dosage units and in one or more dosage forms. A dosage can be administered daily, i.e., consistent administration, or every other day or similar such schedule, i.e., intermittent administration. A dosage can be provided for consistent administration during a period of treatment, for intermittent administration during a period of treatment, or for consistent administration and/or intermittent administration during one or more periods of treatment optionally consistently or intermittently combined with one or more periods of nonadministration or nontreatment depending upon cardiovascular risk level or health needs of the particular individual to which the composition is being administered. Preferably, a dosage of one or more compositions of the present invention is provided as 1 to 4 dosage units of one or more oral dosage forms for consistent administration. However, depending upon cardiovascular risk level, from 1 to as many as 20 dosage units could be administered to a patient daily, recognizing that from 1 to 10 dosage units would be the norm.

A dosage of one or more compositions of the present invention may contain larger amounts of one or more ingredients than that specified herein. The minimum amount of ingredients specified herein reflect the minimum amount of the particular ingredient to be provided upon administration through to the date of product expiration as set forth on the product sale label. However, since one or more of the ingredients may be subject to degradation over time, the dosage must contain larger amounts of those ingredients tending to degrade to compensate for such degradation. By providing larger amounts of ingredients tending to degrade over time in said dosage, one is ensured that even with ingredient degradation, one hundred percent of the ingredient amount specified on the product sale label is provided upon administration of the dosage through to the specified expiration date shown on the product sale label.

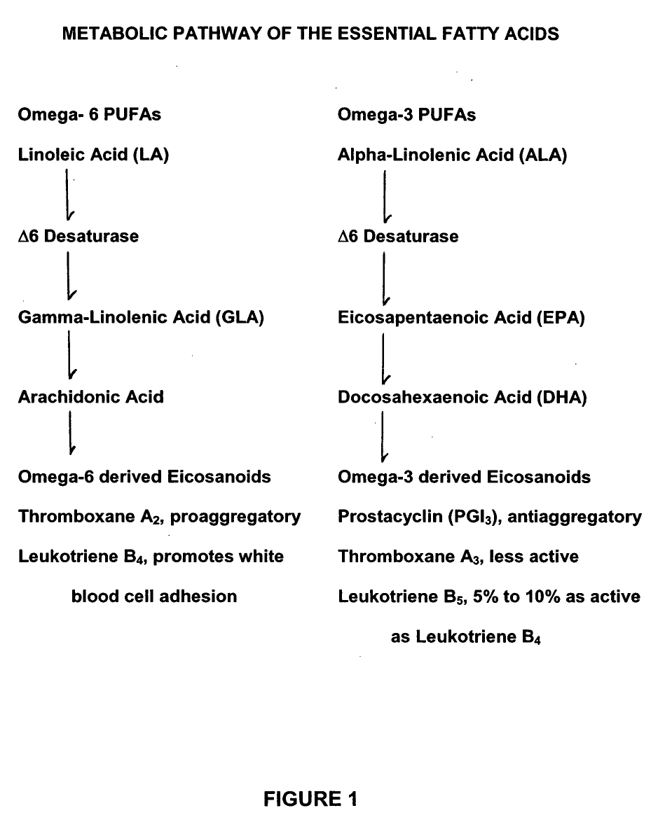

Compositions of the present invention comprise an effective amount of one or more endothelial cell anti-inflammatory agents, one or more nitric oxide generation promoting agents, one or more antioxidant agents, and one or more platelet aggregation lowering agents, to promote cardiovascular health. Suitable endothelial cell anti-inflammatory agents include for example but are not limited to essential fatty acids (EFA) including Omega-3 fatty acids (natural and/or synthetic) such as for example but not limited to eicosapentaenoic acid (EPA), docosahexaenoic acid (DHA) and alpha-linolenic acid (ALA), derivatives of Omega-3 fatty acids, derivatives of EPA, derivatives of DHA, derivatives of ALA, fatty acid compound derivatives such as for example but not limited to phospholipid esters of linolenic acid, ethers of linolenic acid and sterol derivatives of linolenic acid, fatty acid compounds such as but not limited to phosphatidal choline esters of linolenic acid, phosphatidal ether of linolenic acid and sipolsterol ester of linolenic acid, and combinations thereof. Endothelial cell anti-inflammatory agents are present in the subject compositions in an amount sufficient to provide a dosage of about 650 mg or greater to ensure basic dietary needs are met, preferably at about 1.38 g or greater to provide cardiovascular protective effects in addition to basic dietary needs, more preferably at about 2 g or greater to improve blood lipid profile in addition to providing basic dietary needs and cardiovascular protective effects, and most preferably at about 2.68 g or greater to lower blood triglyceride levels, in addition to providing basic dietary needs and cardiovascular protective effects and improving blood lipid profile. Omega-6 fatty acids do not impart the cardiovascular benefits of Omega-3 fatty acids as best illustrated in FIG. 1. Recognizing that Omega-6 fatty acids are often present in fish oil used as a source of EFA, the present compositions contain less than about 150mg, preferably less than about 100 mg and most preferably less than about 50 mg of Omega-6 fatty acids in a dosage. The typical ratio of Omega-6 fatty acids to Omega-3 fatty acids available through diet is high. Based on the standard U.S. diet, a person's standard Omega-6 fatty acid to Omega-3 fatty acid intake ratio is 8.8. It is believed that by lowering this ratio by about 5 percent to about 50 percent, through the use of a nutritional or dietary supplement composition containing Omega-3 fatty acids such as that of the present invention, cardiovascular health is promoted and/or maintained.

Suitable nitric oxide generation promoting agents include for example but are not limited to folic acid (Vitamin B₉, pteroylglutamic acid), folate, precursors of folic acid, precursors of folate, derivatives of folic acid, derivatives of folate, metabolites of folic acid, metabolites of folate, natural isomers of folate such as for example but not limited to (6S)-tetrahydrofolic acid and derivatives thereof, 5-methyl-(6S)-tetrahydrofolic acid and derivatives thereof, 5-formyl-(6S)-tetrahydrofolic acid and derivatives thereof, 10-formyl-(6R)-tetrahydrofolic acid and derivatives thereof, 5,10-methylene-(6R)-tetrahydrofolic acid and derivatives thereof, 5,10-methenyl-(6R)-tetrahydrofolic acid and derivatives thereof, 5-formimino-(6S)-tetrahydrofolic acid and derivatives thereof, 10-formyl-(6RS)-tetrahydrofolic acid and derivatives thereof, 5,10-methylene-(6RS)-tetrahydrofolic acid and derivatives thereof, 5,10-methenyl-(6RS)-tetrahydrofolic acid and derivatives thereof, polyglutamyl and derivatives thereof, and combinations thereof. Natural isomers of folate are the subject matter of U.S. Pat. Nos. 5,997,915 and 6,254,904 incorporated herein by reference in their entirety. Nitric oxide generation promoting agents are present in the subject compositions in an amount sufficient to provide a dosage of about 0.4 mg to about 5 mg to meet dietary needs, preferably in an amount of about 0.8 mg to about 10 mg to provide smooth muscle relaxation, enhanced NO synthase and the like, in addition to dietary needs, more preferably in an amount of about 1.2 mg to about 15 mg to provide enhanced cardiovascular benefits in addition to smooth muscle relaxation, enhanced NO synthase and dietary needs, still more preferably in an amount of about 1.6 mg to about 20 mg for more enhanced cardiovascular benefits in addition to smooth muscle relaxation, enhanced NO synthase and dietary needs, and most preferably in an amount of about 2 mg to about 25 mg for more enhanced cardiovascular benefits such as protection against acute endothelial effects of a fatty meal, in addition to smooth muscle relaxation, enhanced NO synthase and dietary needs.

The use of folic acid to reduce risks associated with cardiovascular diseases is attractive since such use is essentially risk free and relatively inexpensive. There is increasing evidence that high blood or plasma levels of homocysteine are associated with an increased risk of CAD and CD. It is well established that folic acid lowers plasma homocysteine levels. Only a relatively small dosage of folic acid is required to reduce plasma homocysteine levels. However, folic acid has also been shown to exert potential beneficial effects on cardiovascular health through mechanisms independent of homocysteine plasma level lowering as published in Circulation, 2002; 105:22, Arteriosclerosis, Thrombosis, and Vascular Biology, 2001;21:1196 and Nutrition, 2003;1 9:686.

Suitable antioxidant agents include for example but are not limited to Vitamin C (ascorbic acid), natural (RRR-alpha-tocopherol) and synthetic (racemic-alpha-tocopherol) Vitamin E such as but not limited to alpha-tocopherol, beta-tocopherol, gamma-tocopherol, trimethyl tocopheryl acetate, tocopherol succinate, derivatives of alpha-tocopherol, derivatives of beta-tocopherol, derivatives of gamma-tocopherol, derivatives of trimethyl tocopheryl acetate, derivatives of tocopherol succinate, precursors of alpha-tocopherol, precursors of beta-tocopherol, precursors of gamma-tocopherol, precursors of trimethyl tocopheryl acetate, precursors of tocopherol succinate, metabolites of alpha-tocopherol, metabolites of beta-tocopherol, metabolites of gamma-tocopherol, metabolites of trimethyl tocopheryl acetate, metabolites of tocopherol succinate, isomers of alpha-tocopherol, isomers of beta-tocopherol, isomers of gamma-tocopherol, isomers of trimethyl tocopheryl acetate, isomers of tocopherol succinate, derivatives of tocol, derivatives of tocotrienol, agents having Vitamin E functionality and combinations thereof, Vitamin A, flavonoids, carotenoids such as but not limited to beta-carotene, lutein, violaxanthin, neoxanthin, cryptoxanthin, phytofluene, phytoene, lycopene, neurosporene, lactucaxanthin, anhydrolutein, zeaxanthin and combinations thereof, alpha-lipoic acid, phenolic compounds such as but not limited to oligomeric proanthocyanidins, anthocyanosides, ubiqinone or coenzyme-Q 10 (CoQ 10) and combinations thereof. Antioxidant agents are present in the subject compositions in varying amounts depending on the particular antioxidant or antioxidants incorporated into the composition. Antioxidant agent amounts may vary depending on specific amounts required to achieve efficacy and specific toxicity levels for the particular antioxidant agent(s) incorporated into the composition. For example, Vitamin E could be present individually in a sufficient amount to provide about 100 IU to about 2000 IU per dosage, and may optionally be used in combination with other antioxidants. Likewise, Vitamin C could be present individually in a sufficient amount to provide about 100 mg to about 2000 mg per dosage, and may optionally be used in combination with other antioxidants.

Suitable platelet aggregation lowering agents include for example but are not limited to Vitamin B₆, including but not limited to pyridoxine, pyridoxal, pyridoxamine, derivatives of pyridoxine, derivatives of pyridoxal, derivatives of pyridoxamine, precursors of pyridoxine, precursors of pyridoxal, precursors of pyridoxamine, metabolites of pyridoxine, metabolites of pyridoxal, metabolites of pyridoxamine, isomers of pyridoxine, isomers of pyridoxal, isomers of pyridoxamine, agents having Vitamin B₆ functionality and combinations thereof. Platelet aggregation lowering agents are present in the subject compositions in varying amounts depending on the particular platelet aggregation lowering agent incorporated into the composition. For example, Vitamin B₆ could be present individually in an amount sufficient to provide about 12.5 mg to about 500 mg per dosage and may optionally be used in combination with other platelet aggregation lowering agents.

As an example of a composition of the present invention, the endothelial cell anti-inflammatory agent is at least one EFA, the NO generation promoting agent is folic acid, the antioxidant agent is Vitamin E, Vitamin C, Vitamin A, CoQ 10, Beta-carotene or combinations thereof, and the platelet aggregation lowering agent is Vitamin B₆.

As another illustrative example of a composition of the present invention, the endothelial cell anti-inflammatory agent is at least one EFA such as for example EPA, DHA and/or ALA, the NO generation promoting agent is folate, the antioxidant is Vitamin C, and the platelet aggregation lowering agent is Vitamin B₆. Should a combination of EPA and DHA be selected as the endothelial cell anti-inflammatory agent, preferred weight ratios of EPA to DHA preferably would be selected from the following ratios: 100:0; 90-100:0-10; 70-90:10-30; 50-70:30-50; 30-50:50-70; 10-30:70-90 and 0-10:90-100 according to specific patient needs, with a ratio 2.5:1 or above being more preferred. However, depending on specific patient needs, weight ratios of EPA to DHA of 1:2.5 or below are contemplated.

As another illustrative example of a composition of the present invention, the endothelial cell anti-inflammatory agent is at least 250 mg of an EFA, the NO generation promoting agent is at least 0.4 mg of folic acid, the antioxidant agent is at least 10 IU Vitamin E and 100 mg Vitamin C and the platelet aggregation lowering agent is at least 12.5 mg Vitamin B₆ per dosage.

As still another illustrative example of a dosage of a composition of the present invention, the endothelial cell anti-inflammatory agent is about 250 mg to about 20 g of an EFA mixture of EPA and DHA with or without ALA. It is noted that at higher amounts, the endothelial cell anti-inflammatory agent may require multiple dosage units. The NO generation promoting agent is about 0.4 mg to about 25 mg folic acid. The antioxidant agent is about 10 IU to about 2000 IU Vitamin E and/or 100 mg to about 2000 mg Vitamin C and the platelet aggregation lowering agent is about 12.5 mg to about 500 mg Vitamin B₆.

Compositions of the present invention are described in still more detail in the examples provided below. Such examples are provided for illustrative purposes only and are not intended to be limiting to the scope of the present invention.

### EXAMPLE 1

### Method of Making Composition of the Present Invention

Fish oil (850 mg/g Omega-3 fatty acid) 391.5 kg is dispensed into a mixing tank and mixed at 725±50 revolutions per minute (rpm). The mixture is heated until it reaches 45° C.±2° C. Agitation is continued and 38 kg silica is added. The resulting mixture is heated to 50° C. and lecithin-unbleached, NF 14 kg are slowly added. Mixing is continued at 725±50 rpm until the mixture is cooled to 34° C.±2° C. While maintaining a temperature below 37° C., the following is added slowly with continued mixing: Vitamin B₆-pyridoxine HCl, USP 11.25 kg, folic acid 0.975 kg, Omega-3 fatty acid powder (188.4 mg/g) 429 kg and Vitamin E succinate 75 kg. The mixing speed is gradually increased during addition of the powders, to facilitate wetting of the powders. To ensure powders are completely wetted and dispersed, the inside wall of the tank is scraped to prevent build-up of bulk fill material. The speed is adjusted to provide adequate agitation to blend the mixture for 40 minutes while the temperature is maintained below 37° C. One liter of fill is drained from the tank valve and transferred back to the top of mixture during the first five minutes of mixing, at the mid-point in mixing, and during the last five minutes of mixing. The mixer speed is adjusted to 725±50 rpm as the mixture is cooled to 30° C.±2° C. As the mixture is cooled, the inside wall of the tank is scraped to prevent build up of bulk fill material. Once the temperature reached 30° C.±2° C., the mixers are stopped. The entire mixture/blend is transferred to 500 series receivers through a Fryma deaeration unit. A finished fill weigh-up using ACF-404RX is performed to record weighing information.

### EXAMPLE 2

### Composition Dosage of the Present Invention

A dosage of a composition of the present invention is provided in the form of two gel capsules of differing formulations as specified in Table 1 below.

TABLE 1

| | |
|---|---|
| Capsule 1 | Capsule 2 |

| | | | | |
|---|---|---|---|---|
| EPA | 300 | mg | 350 | mg |
| DHA | 100 | mg | 100 | mg |
| Linolenic Acid | 50 | mg | 100 | mg |
| Folic Acid | 1 | mg | 0 | |
| Vitamin B₆ | 12.5 | mg | 0 | |
| Vitamin E | 10 | IU | 90 | IU |

### EXAMPLE 3

### Composition Dosage of the Present Invention

A dosage of a composition of the present invention is provided in the form of one tablet and one gel capsule of differing formulations as specified in Table 2 below.

TABLE 2

| | |
|---|---|
| Tablet | Capsule |

| | | | | |
|---|---|---|---|---|
| EPA/DHA | 0 | | 400 | mg |
| ALA | 0 | | 100 | mg |
| Vitamin E | 0 | | 100 | IU |
| Folic Acid | 1 | mg | 0 | |
| Vitamin B₆ | 12.5 | mg | 0 | |
| Vitamin D | 400 | IU | 0 | |
| Calcium | 600 | mg | 0 | |

### EXAMPLE 4

### Composition Dosage of the Present Invention

A dosage of a composition of the present invention is provided in the form of two tablets of differing formulations and two gel capsules of like formulation for use in an “uneven administration” regimen as specified in Table 3 below.

TABLE 3

| | |
|---|---|
| Morning Tablet | Evening Tablet |

| | | | | |
|---|---|---|---|---|
| Vitamin D | 200 | IU | 600 | IU |
| Calcium | 400 | mg | 600 | mg |
| Vitamin C | 25 | mg | 25 | mg |
| Folic Acid | 2.5 | mg | 1.5 | mg |
| Vitamin B6 | 25 | mg | 12.5 | mg |

| | Gel Capsules* each contain | |
|---|---|---|
| EPA | 300 | mg |
| DHA | 100 | mg |
| ALA | 100 | mg |
| Vitamin E | 150 | IU |
| Linoleic Acid | 35 | mg |

*Preferably, one or more gel capsules are taken with the morning tablet and/or one or more gel capsules are taken with the evening tablet.

Compositions of the present invention may be used in the promotion and/or maintenance of cardiovascular health of a patient. A method of promoting and/or maintaining cardiovascular health of a patient includes assessing the cardiovascular health of the patient; determining proper stratification or categorization of the patient based on the cardiovascular health of the patient; and administering a composition of the present invention of a formulation suitable based on the stratification or categorization of the patient.

In accordance with the present invention, a patient's cardiovascular health is assessed or evaluated using a recognized risk factor evaluation model as discussed above, in order to stratify or categorize the patient into one of five levels of cardiovascular risk. The Framingham Study noted above designates five levels of cardiovascular risk as “very low”, “low”, “moderate”, “high” and “very high”. The Cardiovascular Risk Assessment Algorithm noted above designates 1^{st} through 5^{th} quintiles of cardiovascular risk. The European Coronary Risk Chart noted above designates particular cardiovascular risk factors for categorizing patients, i.e., “no family history”, “family history”, “age 40-55 years”, “CAD” and “very high transglycerides” (TGs). Each of these risk factor evaluation models and designations are set forth below in Table 4.

The present invention provides a method for utilizing a patient's specific cardiovascular risk level to determine the proper composition formulation to administer to that particular patient. Preferred embodiments of compositions of the present invention have been specifically formulated based on a large body of clinical data generated by cross-sectional epidemiologic studies, as well as prospective observational studies, to promote cardiovascular health based on a patient's particular cardiovascular risk level. A specific formulation of a composition of the present invention may be administered as an effective dosage or the formulation may be multiplied prior to administration as an effective dosage depending on a patient's cardiovascular risk level, i.e., very low, low, moderate, high or very high, irrespective of which risk factor evaluation model is used. Categorization or stratification of patients is relatively simple. However, should an error occur with regard to the stratification or categorization of a patient, any of the five levels of dosing would still be beneficial to the patient.

To describe the present invention in more detail, a specific formulation of a composition of the present invention is provided for each cardiovascular risk level. For a first, “very low” cardiovascular risk level, a preferred composition would comprise about 250 mg to about 1500 mg of EPA, DHA and ALA, about 0.4 mg to about 5 mg of folic acid, about 10 IU to about 400 IU of Vitamin E and about 12.5 mg to about 100 mg of Vitamin B₆ per dosage.

For a second, “low” cardiovascular risk level, a preferred composition would comprise about 500 mg to about 3000 mg of EPA, DHA and ALA, about 0.8 mg to about 10 mg folic acid, about 20 IU to about 800 IU Vitamin E and about 25 mg to about 200 mg Vitamin B₆ per dosage. This dietary or nutritional supplement composition provides at least the recommended dietary requirement for EFA Omega 3 as is generally agreed to by expert panel guidelines and the literature.

For a third, “moderate” cardiovascular risk level, a preferred composition would comprise about 750 mg to about 4500 mg of EPA, DHA and ALA, about 1.2 mg to about 15 mg folic acid, about 30 IU to about 1200 IU Vitamin E and about 37.5 mg to about 300 mg Vitamin B₆ per dosage. This supplement provides cardio-protective benefits.

For a fourth, “high” cardiovascular risk level, a preferred composition would comprise about 1000 mg to about 6000 mg of EPA, DHA and ALA, about 1.6 mg to about 20 mg folic acid, about 40 IU to about 1600 IU Vitamin E and about 50 mg to about 400 mg Vitamin B₆ per dosage, to provide lipo-profile enhancement.

Finally, for a fifth, “very high” cardiovascular risk level, a preferred composition would comprise about 1250 mg to about 7500 mg of EPA, DHA and ALA, about 2 mg to about 25 mg folic acid, about 50 IU to about 2000 IU Vitamin E and about 67.5 mg to about 500 mg Vitamin B₆ per dosage, to provide a reduction of blood triglyceride levels.

Each of the above-described compositions has been carefully formulated to achieve specific preventative and/or therapeutic goals at each specific cardiovascular risk level. In formulating compositions of the present invention to meet the needs of each cardiovascular risk level as described above, “stepwise” increasing of each ingredient of a single composition formulation dosage, such as for example but not limited to doubling, tripling, quadrupling and quintupling the dosage, is preferred. In the case that compositions of the present invention are formulated stepwise, the composition may be administered by providing a dosage or a “base formulation” to a person identified in a first, “very low” cardiovascular risk level, i.e., level 1. A person identified in a second, “low” cardiovascular risk level, i.e., level 2, would be administered a dosage of two times the base formulation. A person identified in a third, “moderate” cardiovascular risk level, i.e., level 3, would be administered a dosage of three times the base formulation. A person identified in a fourth, “high” cardiovascular risk level, i.e., level 4, would be administered a dosage of four times the base formulation. A person identified in a fifth, “very high” cardiovascular risk level, i.e., level 5, would be administered a dosage of five times the base formulation. It is recognized that some physicians may use a risk factor evaluation model that differs from the Framingham Model having five stratification levels. For example, should a physician apply a risk factor evaluation model having only three stratification or categorization levels, the physician my administer a dosage or a “base formulation” to a person identified in a first, “low” cardiovascular risk level, a dosage three times the base formulation to a person identified in a second, “moderate” cardiovascular risk level and a dosage five times the base formulation to a person identified in a third, “high” cardiovascular risk level. Accordingly, the invention contemplates increasing the dosage as a patient's cardiovascular risk level increases regardless of which risk factor evaluation model applied. With each successive increase in the dosage amount, the composition provides all the benefits of the lesser dosage amount(s) in addition to providing the additional benefits noted. Also, by contrast, the dosage amount may be decreased as a patient's cardiovascular risk level decreases. Such decrease in the dosage amount would be implemented in the same stepwise fashion as used in increasing the dosage amount as described in detail above.

It is noted that in each composition formulation provided above, the individual components or ingredients, such as for example Vitamin E, are used for illustrative purposes only. The individual components or ingredients can be replaced or supplemented by any other suitable component as described in detail above. For example, Vitamin E, an antioxidant, may be replaced or supplemented by any other suitable antioxidant agent including but not limited to Vitamin C, Vitamin A, Beta-carotene or any combination thereof.

In another preferred embodiment, illustrated in Table 4 below, specific composition formulations are provided for illustrative purposes only. The risk factor evaluation model utilized has five stratification or categorization cardiovascular risk levels based on a patient's particular cardiovascular health.

TABLE 4
COMPOSITION FORMULATION PER RISK LEVEL

| | RISK MODELS | | | PRODUCT | | | | | | Intended |
|---|---|---|---|---|---|---|---|---|---|---|
| | Framingham | Level | Rationale | EFA | | E | FA | B₆ | | Benefits |
| 1 | Very Low | 1^{st} Quintile | No CV History | 500 | mg | 50 IU | .4 mg | 12.5 | mg | Supplements |
| | | | | | | | | | | CV Nutrition |
| 2 | Low | 2^{nd} Quintile | Familial History | 650 | mg | 100 IU | 1 mg | 25 | mg | Provide U.S. |
| | | | | | | | | | | Recommended |
| | | | | | | | | | | Guideline |
| 3 | Moderate | 3^{rd} Quintile | Age 40-55 | 1.38 | g | 200 IU | 2 mg | 50 | mg | Cardio- |
| | | | | | | | | | | protective |
| 4 | High | 4^{th} Quintile | CV Disease | 2 | g | 300 IU | 3 mg | 75 | mg | Lipid profile |
| | | | | | | | | | | enhancement |
| 5 | Very High | 5^{th} Quintile | Very High | 2.68 | G | 400 IU | 4 mg | 100 | mg | Triglyceride |
| | | | Triglyceride | | | | | | | reduction |
| | | | Levels | | | | | | | |

EFA = Essential Fatty Acid
E = Vitamin E
FA = Fatty Acid
B₆ = Vitamin B₆
CV = Cardiovascular

Composition formulations provided in Table 4 above could be produced in a number of ways. In one embodiment, the composition would be available in two differing formulations. A first formulation comprising a dosage of about 0.5 g EFA, about 50 IU Vitamin E, about 0.4 mg folic acid and about 12.5 mg Vitamin B₆, would be prescribed and/or administered by a healthcare provider as a dietary supplement to a patient in a very low risk level according to at least one of the risk factor evaluation models noted above. A second formulation comprising a dosage of about 0.67 g EFA, about 100 IU Vitamin E, about 1 mg folic acid and about 25 mg Vitamin B₆, would be prescribed and/or administered by a healthcare provider to a patient in the next lowest risk level. This same second formulation could then be prescribed and/or administered by a healthcare provider in accordance with the following: two times the second formulation dosage for a moderate risk category, three times the second formulation dosage for a high risk category and four times the second formulation dosage for a very high risk category.

An additional preferred embodiment of the present invention includes a composition comprising a dosage of at least about 250 mg EFA, about 0.4 mg folic acid, at least about 10 IU Vitamin E, about 100 mg Vitamin C, and at least about 12.5 mg Vitamin B₆. Another preferred embodiment includes a composition comprising a dosage of about 250 mg to about 20 g of a mixture of EPA and DHA with or without ALA, about 0.4 mg to about 0.25 mg folic acid, about 10 IU to about 2000 IU Vitamin E, optionally, about 100 mg to about 2000 mg Vitamin C and about 12.5 mg to about 500 mg Vitamin B₆. It is noted that at higher amounts it may be necessary to provide the EFAs in multiple dosage units. For example, EFAs in powdered form may be added to formulation oils to maximize the EFA dosage while maintaining a reasonably sized dosage form. It is also noted that as used herein, “about” is intended to mean “plus or minus five percent”.

Compositions of the present invention may optionally include at least one other B complex vitamin in combination with Vitamin B₆. B complex vitamins that are useful for purposes of the present invention include those selected from the group consisting of Vitamin B₁ (thiamine), Vitamin B₂ (riboflavin), Vitamin B₁₂ family (cyanocobalamin and the like), niacin (nicotinic acid and nicotinamide), pantothenic acid, biotin, choline and combinations thereof. Compositions of the present invention may also be supplemented with other vitamins and/or minerals as are known in the art. Also, compositions of the present invention may include artificial sweeteners, aromatics and/or flavoring agents as are well known in the art.

Optionally, compositions of the present invention may be provided in combination with calcium, Vitamin D, natural or synthetic analogs of Vitamin D, 1, 25-dihydroxycholecalciferol or mixtures thereof. Unfortunately, EFA or other components or ingredients and calcium may not be compatible in a product, given the disparate moisture levels of the components. Accordingly, EFA and calcium components may be provided in a strip pack that provides the EFA containing components of the present invention in a separate dosage unit from that of the calcium containing components.

Compositions of the present invention may be used independently to promote and/or maintain cardiovascular health or used in combination with one or more other compositions used in the treatment of various other disease states common to aging and/or a health deteriorating condition. As examples not intended to be limiting, compositions of the present invention may be used independently or in combination with one or more treatments for cardiovascular diseases such as but not limited to anti-arrhythmias, hypertension, and venous thrombosis, for misfolding diseases such as but not limited to Parkinson's disease, Kreutzfeld Jacob's disease, renal amyloidosis and Huntington's chorea, for degenerative diseases affecting cartilage, for neurological diseases such as but not limited to Alzheimer's disease and dementia, for topical conditions such as but not limited to sunburn and topical absorption needs, for diseases affecting behavior such as but not limited to depression, mania, schizophrenia, attention deficit disorder, attention deficit hyperactivity disorder, obesity and postpartum depression, for reproductive conditions such as but not limited to male infertility, pre-eclampsia and low birth weight, for diseases associated with inflammation such as but not limited to mucositis, atherosclerosis, inflammatory bowel disease, cystic fibrosis and psoriasis, for autoimmune diseases such as but not limited to rheumatoid, systemic lupus erythematosus, glomeruloscierosis and pulmonary fibrosis and for ophthalmic diseases such as but not limited to macular degeneration and glaucoma. Compositions of the present invention may also be used in combination with HMG CoA reductase inhibitors not limited to anti-inflammatory agents such as but not limited to non-steroidal anti-inflammatory drugs (NSAIDs), cycloxygenase-2 drugs (COX-2) and nitrate drugs, statin drugs, antiplatelet drugs, homocysteine lowering drugs, and fibrates not limited to fibric acid, gemfibrozil, fenofibrate and derivatives thereof.

Compositions of the present invention in various dosages may be marketed in blister packaging designed for various risk levels as described above. Compositions of the present invention may be provided as a single dosage or as multiple dosages in one or more dosage units and in one or more dosage forms. Compositions of the present invention may also be color coded for convenience.

Packaging of compositions of the present invention is preferably accomplished using a storage stable disposable dispensing container which provides optimal therapeutic and/or nutritional support to a human or other animal by increasing compliance with a dosage regimen and facilitating administration of possible storage-incompatible substances. Suitable packaging includes various types of blister-type packaging. Blister-type packaging is characterized by a plurality of single compartments referred to herein as “recesses”. Each recess accommodates a dosage unit and isolates that dosage unit from other dosage units. In this manner, the biologically-active substance within each dosage unit will not come into contact with the biologically-active substance of the other dosage units, despite being in close proximity to other dosage units in the blister packaging. This arrangement, particularly when incorporating day and time indications corresponding to said recesses, eases simultaneous administration of storage-incompatible substances as required by a complex dosing regimen which provides optimal therapeutic support.

Disposable pharmaceutical packaging for dispensing medicaments used to improve patient compliance, have been previously disclosed. One type of pharmaceutical dispensing packaging arranges medicaments separately within individual recesses upon a planar card to form blister-type packaging. An example of such a package can be found in Knudsen, U.S. Pat. No. 4,295,567, incorporated herein in its entirety by reference. Knudsen discloses a pharmaceutical dispensing container which holds two dosage units for symptomatic treatment of respiratory tract disorders.

Pharmaceutical dispensing packaging wherein medicaments are arranged separately within individual recesses upon a planar card, may further be inserted into a container designed to protect and/or otherwise further precipitate dispensing of the medication is also known in the art. An example of such a package can be found in Leonard et al., U.S. Pat. No. 4,376,849 incorporated herein in its entirety by reference. Leonard et al. describe a method and apparatus to store and aid in dispensing calendar-oriented drugs. The apparatus is comprised of a carrier containing a plurality of pill-containing enclosures, which are arranged in rows. Numerical and/or alphanumerical indicia are associated with the enclosures so that each enclosure is associated with only one day in a calendar month. One or more additional enclosures in different rows may also be associated with the same calendar date. Corresponding indicia on the reverse side of the carrier aid in the determination of which enclosure(s) to open. The package also provides a visual indication of calendar days for which pills have not been used by the patient and in this way provides patient compliance information to the physician prescribing such drugs. This dispensing apparatus is particularly suited to the administration of calendar-oriented prescription drugs for the treatment of menopausal symptoms.

Packaging for compositions of the present invention may be made by techniques well known and readily available to persons of ordinary skill in the art. Various types of blister packaging may be used, without limitation. For example, one type of blister packaging that may be used is a “push-through” pack. Push-through packs have recesses with a lid of aluminum foil or an aluminum foil laminate. Aluminum foil is a preferred material for the lids on push-through packs as the thickness of the material employed requires relatively little force for rupture thereof. Consequently, the energy for penetration is low since aluminum exhibits essentially no elasticity. The base of the push-through pack may be made of plastic, such as for example but not limited to polyvinyl chloride, polyamides, polyolefins, polyesters and laminates or multi-layered materials containing at least one of these materials and, if desired, also containing an aluminum foil. Other types of push-through packs may feature a base covered by a foil lid. The foil lid may cover the whole of the base area and is usefully provided with a line of weakness in the region of each recess, or alternatively each recess may be covered with an individual lid segment. If having a line of weakness, the lid may be opened by splitting the same at the line of weakness. If covered with an individual lid segment, each lid segment may be equipped with a tab for gripping. This tab enables the individual recess to be exposed by pulling and separating the lid segment from the base. The base and the lid may be made of any of the above materials, whereby plastic laminates may also be employed for the lid materials.

Bases of suitable blister packs may be embossed, cast, deep drawn or vacuum formed out of plastic, plastic laminates, plastic/paper laminates, plastic/metal foil laminates and the like. Non-limiting exemplary suitable plastics for bases are films and film laminates containing polyvinyl chloride, polyamides, polyolefins, polyesters, polycarbonates and combinations thereof. The bases may also feature a barrier layer against gases and vapors. Such barrier layers may be a metal foil such as an aluminum foil embedded in a plastic laminate or usefully ceramic layers or metallic layers embedded between two plastic layers. Ceramic layers may be produced by evaporating metals, oxides or nitrides of aluminum, silicon and other metals and semimetals in vacuum and depositing the substances on a plastic substrate. These methods are known as chemical vapor deposition and physical vapor deposition or sputtering. The ceramic layers may contain aluminum oxides or silicon oxides or may be mixtures of various oxides. The ceramic layers may also be mixed with metals such as silicon or aluminum. Metal layers may be created by evaporating metals in vacuum and depositing metal layers, such as for example but not limited to aluminum layers, on a plastic substrate. The plastic substrate may be a plastic film or a plastic base made of the above mentioned plastics. As a general rule, the lid material for a push-through pack is an aluminum foil or a laminate containing aluminum foil. It has been proposed to replace the aluminum foil with a plastic that exhibits low elasticity and poor elongation properties. Such plastics may be obtained when large amounts of filler materials are added to the plastic. Such a container would make it possible to easily sort waste material, for example, by separating metal and plastics. Plastics and plastic laminates could also be employed for blister packs with peel back lid material.

Packaging for compositions of the present invention preferably features between 4 and 28 recesses in the form of cups or dishes, without limitation. The recesses may be surrounded by a shoulder, said shoulders together forming an interconnected flat plane. The bases are prepared, for example, as a strip with the contents in recesses. The base strip is brought together with a lid material, in particular a foil lid form, likewise in the form of a strip. The lid foil covers the base completely and by sealing or adhesive bonding is joined to the base at the shoulders. The lid foil may be sealed or adhesively bonded to the shoulders over the whole area or, by choosing a special sealing tool or bonding pattern for the purpose. This sealing or bonding may be only partial. Next, the strips of lidded base may be cut to the desired size. This may be performed using a stamping tool. At the same time, the blister packaging may be formed to have outer contours. It is also possible to provide weaknesses in the lid material or in the base to allow the blister package to be bent or to create lid segments, to make removal of the lid segment and removal of the contents possible.

Packaged compositions of the present invention may be in one or more dosage forms such as for example but not limited to a tablet, caplet, capsule, gel capsule, chew tablet, lozenge and troche, nutritional bar or food item, soft chew, reconstitutable powder or shake, sprinkle, semi-solid sachet or the like. Any tablet dosage form may be either chewable or compressed. The preferred solid dosage form for purposes of the present invention is a gel capsule. However, compositions of the present invention could likewise be incorporated into a food product or a powder for mixing with a liquid.

Although any solid dosage form can be used to provide a dosage of one or more compositions of the present invention, preferred dosage forms include a single gel capsule, two gel capsules or one gel capsule and one caplet or tablet. For example, one capsule may contain from about 40% to about 60% of the EFAs and a second capsule may contain the remainder of the EFAs. The remaining vitamins and minerals may be divided between the two capsules as desired. Alternatively, a dosage may be provided in one gel capsule and one tablet. For example, the EFAs may be provided in a gel capsule, while the remaining ingredients may be provided in a tablet. It is critical that a sufficient amount of EFAs be administered to the patient. Accordingly, at higher amounts of EFAs, it may be necessary for the patient to take more than one dosage unit.

In the event that the composition formulation is provided in the dosage form of one gel capsule and one tablet, the invention contemplates administering such dosage forms several times daily, including twice daily. In that event it is preferred that the gel capsule contain EFAs and Vitamin E and the tablet contain other vitamins and minerals. More preferred would be a system wherein a tablet administered in the morning contain different amounts of vitamins and minerals than that taken in the evening.

In practice, to use compositions of the present invention, a health care professional would take a patient history including age, cigarette smoking habits, hypertension, lipid abnormalities, family history, diabetes and the prevalence of CAD and/or CD. The professional then uses the history in combination with a commercially available risk factor evaluation model chart to stratify or classify the patient into one of five cardiovascular risk levels noted above. The composition formulation for that risk level is then prescribed to the patient, typically for administration in a 24 hour period. In some cases, a healthcare professional will actually administer the supplement to the patient.

Compositions of the present invention could also be prescribed for the treatment of genetic diseases including but not limited to cystic fibrosis, inflammatory diseases including but not limited to atherosclerosis, cirrhosis, mucositis, chronic pancreatitis, asthma and inflammatory bowel disease, or diseases of natural aging or a health deteriorating condition.

Also, compositions disclosed herein may be used to treat autoimmune diseases including but not limited to rheumatoid arthritis, glomerulosclerosis, pulmonary fibrosis and systemic lupus erythematosis.

In another alternative embodiment, the products disclosed herein may be administered to perimenopausal or menopausal women to maintain or promote cardiovascular health.

In another alternative embodiment, the products of the present invention are administered to teenagers to maintain or promote cardiovascular health.

In still another embodiment of the present invention, there is disclosed a method for determining a patient's cardiovascular risk level starting point through stratifying or categorizing a patient's cardiovascular risk factors into a risk level, prescribing the appropriate composition dosage based on risk level, and moving the patient to a more favorable risk level within a prescribed period of time called the endpoint. Preferably, the risk level improvement will be one risk level within about 2 to about 6 months, more preferably one risk level within about three months.

In accordance with the above detailed description, the present invention provides dietary or nutritional supplement composition in one or more dosage forms for providing an effective amount of Omega-3 fatty acids to be beneficial in the treatment or prevention of CAD and/or CD. The invention contemplates administering large doses of Omega-3 fatty acids to a patient, while minimizing Omega-6 fatty acids to improve a patient's Omega-6 fatty acid to Omega-3 fatty acid ratio as noted above. Preferably a patient's Omega-6 fatty acid to Omega-3 fatty acid ratio is lowered using compositions of the present invention by about 5 percent to about 50 percent.

Having described the present invention in detail, those skilled in the art will appreciate that modifications may be made to the invention without departing from the spirit and scope thereof. Therefore, it is not intended that the scope of the invention be limited to the specific embodiments described herein. Rather, it is intended only that the appended claims determine the scope of the invention.

We claim:

1. A composition comprising:
an effective amount of one or more endothelial cell anti-inflammatory agents;
an effective amount of one or more nitric oxide generation promoting agents;
an effective amount of one or more antioxidant agents; and
an effective amount of one or more platelet aggregation lowering agents, to promote or maintain health.

2. A cardiovascular health promoting or maintaining composition comprising:
an effective amount of one or more endothelial cell anti-inflammatory agents;
an effective amount of one or more nitric oxide generation promoting agents;
an effective amount of one or more antioxidant agents; and
an effective amount of one or more platelet aggregation lowering agents, in one or more dosage forms.

3. A composition for treatment of a cardiovascular disease comprising:
an effective amount of one or more endothelial cell anti-inflammatory agents;
an effective amount of one or more nitric oxide generation promoting agents;
an effective amount of one or more antioxidant agents; and
an effective amount of one or more platelet aggregation lowering agents, in one or more dosage forms for treatment of a cardiovascular disease.

4. A cardiovascular health promoting or maintaining composition comprising:
an effective amount of one or more endothelial cell anti-inflammatory agents;
an effective amount of one or more nitric oxide generation promoting agents;
an effective amount of one or more antioxidant agents; and
an effective amount of one or more platelet aggregation lowering agents, in one or more dosage forms to prevent, stabilize, reverse or treat coronary artery disease or cerebrovascular disease.

5. The composition of claim 1, 2, 3 or 4 wherein said one or more endothelial cell anti-inflammatory agents are selected from the group consisting of natural Omega-3 fatty acids and synthetic Omega-3 fatty acids.

6. The composition of claim 1, 2, 3 or 4 wherein said one or more endothelial cell anti-inflammatory agents are selected from the group consisting of eicosapentaenoic acid, docosahexaenoic acid, alpha-linolenic acid, derivatives of eicosapentaenoic acid, derivatives of docosahexaenoic acid, derivatives of alpha-linolenic acid and fatty acid compound derivatives.

7. The composition of claim 1, 2, 3 or 4 wherein said one or more endothelial cell anti-inflammatory agents are selected from the group consisting of eicosapentaenoic acid, docosahexaenoic acid, alpha-linolenic acid, derivatives of eicosapentaenoic acid, derivatives of docosahexaenoic acid, derivatives of alpha-linolenic acid, phospholipids esters of linolenic acid, ethers of linolenic acid, sterol derivatives of linolenic acid and fatty acid compounds.

8. The composition of claim 1, 2, 3 or 4 wherein said one or more endothelial cell anti-inflammatory agents are selected from the group consisting of eicosapentaenoic acid, docosahexaenoic acid, alpha-linolenic acid, derivatives of eicosapentaenoic acid, derivatives of docosahexaenoic acid, derivatives of alpha-linolenic acid, phospholipids esters of linolenic acid, ethers of linolenic acid, sterol derivatives of linolenic acid, phosphatidal choline esters of linolenic acid, phosphatidal ether of linolenic acid and sipolsterol ester of linolenic acid.

9. The composition of claim 1, 2, 3 or 4 wherein said one or more nitric oxide generation promoting agents are selected from the group consisting of folic acid, folate, precursors of folic acid, precursors of folate, derivatives of folic acid, derivatives of folate, metabolites of folic acid, metabolites of folate and natural isomers of folate.

10. The composition of claim 1, 2, 3 or 4 wherein said one or more nitric oxide generation promoting agents are selected from the group consisting of folic acid, folate, precursors of folic acid, precursors of folate, derivatives of folic acid, derivatives of folate, metabolites of folic acid, metabolites of folate, (6S)-tetrahydrofolic acid, derivatives of (6S)-tetrahydrofolic acid, 5-methyl-(6S)-tetrahydrofolic acid, derivatives of 5-methyl-(6S)-tetrahydrofolic acid, 5-formyl-(6S)-tetrahydrofolic acid, derivatives of 5-formyl-(6S)-tetrahydrofolic acid, 10-formyl-(6R)-tetrahydrofolic acid, derivatives of 10-formyl-(6R)-tetrahydrofolic acid, 5,10-methylene-(6R)-tetrahydrofolic acid, derivatives of 5,10-methylene-(6R)-tetrahydrofolic acid, 5,10-methenyl-(6R)-tetrahydrofolic acid, derivatives of 5,10-methenyl-(6R)-tetrahydrofolic acid, 5-formimino-(6S)-tetrahydrofolic acid, derivatives of 5-formimino-(6S)-tetrahydrofolic acid, 10-formyl-(6RS)-tetrahydrofolic acid, derivatives of 10-formyl-(6RS)-tetrahydrofolic acid, 5,10-methylene-(6RS)-tetrahydrofolic acid, derivatives of 5,10-methylene-(6RS)-tetrahydrofolic acid, 5,10-methenyl-(6RS)-tetrahydrofolic acid, derivatives of 5,10-methenyl-(6RS)-tetrahydrofolic acid, polyglutamyl and derivatives of polyglutamyl.

11. The composition of claim 1, 2, 3 or 4 wherein said one or more antioxidant agents are selected from the group consisting of ascorbic acid, alpha-tocopherol, trimethyl tocopheryl acetate, tocopherol succinate, Vitamin A, flavonoids, caretenoids, alpha-lipoic acid, phenolic compounds and CoQ 10.

12. The composition of claim 1, 2, 3 or 4 wherein said one or more antioxidant agents are selected from the group consisting of ascorbic acid, alpha-tocopherol, trimethyl tocopheryl acetate, alpha-tocopherol succinate, Vitamin A, flavonoids, beta-carotene, lutein, violaxanthin, neoxanthin, cryptoxanthin, phytofluene, phytoene, lycopene,neurosporene, lactucaxanthin, anhydrolutein, zeaxanthin, alpha-lipoic acid, oligomeric proanthocyanidins, anthocyanosides and CoQ 10.

13. The composition of claim 1, 2, 3 or 4 wherein said one or more antioxidant agents are selected from the group consisting of natural alpha-tocopherol, synthetic alpha-tocopherol, beta-tocopherol, gamma-tocopherol, trimethyl tocopheryl acetate, tocopherol succinate, derivatives of alpha-tocopherol, derivatives of beta-tocopherol, derivatives of gamma-tocopherol, derivatives of trimethyl tocopheryl acetate, derivatives of tocopherol succinate, precursors of alpha-tocopherol, precursors of beta-tocopherol, precursors or gamma-tocopherol, precursors of trimethyl tocopheryl acetate, precursors of tocopherol succinate, metabolites of alpha-tocopherol, metabolites of beta-tocopherol, metabolites of gamma-tocopherol metabolites of trimethyl tocopheryl acetate, metabolites of tocopherol succinate, isomers of alpha-tocopherol, isomers of beta-tocopherol, isomers of gamma-tocopherol, isomers of trimethyl tocopheryl acetate, isomers of tocopherol succinate, derivatives of tocol, derivatives of tocotrienol, agents having Vitamin E functionality, Vitamin A, Vitamin C, flavonoids, beta-carotene, lutein, violaxanthin, neoxanthin, cryptoxanthin, phytofluene, phytoene, lycopene,neurosporene, lactucaxanthin, anhydrolutein, zeaxanthin, alpha-lipoic acid, oligomeric proanthocyanidins, anthocyanosides and CoQ 10.

14. The composition of claim 1, 2, 3 or 4 wherein said one or more platelet aggregation lowering agents are selected from the group consisting of Vitamin B₆ and agents having Vitamin B₆ functionality.

15. The composition of claim 1, 2, 3 or 4 wherein said one or more platelet aggregation lowering agents are selected from the group consisting of pyridoxine, pyridoxal, pyridoxamine, derivatives of pyridoxine, derivatives of pyridoxal, derivatives of pyridoxamine, precursors of pyridoxine, precursors of pyridoxal, precursors of pyridoxamine, metabolites of pyridoxine, metabolites of pyridoxal, metabolites of pyridoxamine, isomers of pyridoxine, isomers of pyridoxal, isomers of pyridoxamine and agents having Vitamin B₆ functionality.

16. The composition of claim 1, 2, 3, 4, 5, 6, 7 or 8 wherein said one or more endothelial cell anti-inflammatory agents are present in an amount of about 650 mg or greater.

17. The composition of claim 1, 2, 3, 4, 5, 6, 7 or 8 wherein said one or more endothelial cell anti-inflammatory agents are present in an amount of about 1.38 g or greater.

18. The composition of claim 1, 2, 3, 4, 5, 6, 7 or 8 wherein said one or more endothelial cell anti-inflammatory agents are present in an amount of about 2.0 g or greater.

19. The composition of claim 1, 2, 3, 4, 5, 6, 7 or 8 wherein said one or more endothelial cell anti-inflammatory agents are present in an amount of about 2.68 g or greater.

20. The composition of claim 1, 2, 3, 4, 9 or 10 wherein said one or more nitric oxide generation promoting agents are present in an amount of about 0.4 mg to about 5 mg.

21. The composition of claim 1, 2, 3, 4, 9 or 10 wherein said one or more nitric oxide generation promoting agents are present in an amount of about 0.8 mg to about 10 mg.

22. The composition of claim 1, 2, 3, 4, 9 or 10 wherein said one or more nitric oxide generation promoting agents are present in an amount of about 1.2 mg to about 15 mg.

23. The composition of claim 1, 2, 3, 4, 9 or 10 wherein said one or more nitric oxide generation promoting agents are present in an amount of about 1.6 mg to about 20 mg.

24. The composition of claim 1, 2, 3, 4, 9 or 10 wherein said one or more nitric oxide generation promoting agents are present in an amount of about 2.0 mg to about 25 mg.

25. The composition of claim 1, 2, 3, 4, 11, 12 or 13 wherein said one or more antioxidant agents are present with about 100 IU to about 2000 IU as Vitamin E.

26. The composition of claim 1, 2, 3, 4, 11, 12 or 13 wherein said one or more antioxidant agents are present with about 100 mg to about 2000 mg as Vitamin C.

27. The composition of claim 1, 2, 3, 4, 14 or 15 wherein said one or more platelet aggregation lowering agents are present with about 12.5 mg to about 500 mg as Vitamin B₆.

28. The composition of claim 1, 2, 3, 4, 14 or 15 wherein said one or more platelet aggregation lowering agents are present with about 12.5 mg to about 500 mg as an agent having Vitamin B₆ functionality.

29. A method of making a composition comprising:
combining an effective amount of one or more endothelial cell anti-inflammatory agents; an effective amount of one or more nitric oxide generation promoting agents; an effective amount of one or more antioxidant agents and an effective amount of one or more platelet aggregation lowering agents; to promote or maintain health.

30. A method of making a cardiovascular health promoting or maintaining composition comprising:
combining an effective amount of one or more endothelial cell anti-inflammatory agents; an effective amount of one or more nitric oxide generation promoting agents; an effective amount of one or more antioxidant agents; and
an effective amount of one or more platelet aggregation lowering agents, in one or more dosage forms.

31. A method of making a composition for treatment of a cardiovascular disease comprising:
combining an effective amount of one or more endothelial cell anti-inflammatory agents; an effective amount of one or more nitric oxide generation promoting agents; an effective amount of one or more antioxidant agents; and
an effective amount of one or more platelet aggregation lowering agents, in one or more dosage forms for treatment of a cardiovascular disease.

32. A method of making a cardiovascular health promoting or maintaining composition comprising:
combining an effective amount of one or more endothelial cell anti-inflammatory agents; an effective amount of one or more nitric oxide generation promoting agents; an effective amount of one or more antioxidant agents; and
an effective amount of one or more platelet aggregation lowering agents, in one or more dosage forms to prevent, stabilize, reverse or treatment coronary artery disease or cerebrovascular disease.

33. A method of using the composition of claim 1, 2, 3, or 4 comprising:
administering said composition to a human or other animal one or more dosage forms using consistent administration or intermittent administration.

34. The method of claim 29, 30, 31 or 32 wherein said one or more endothelial cell anti-inflammatory agents are selected from the group consisting of natural Omega-3 fatty acids and synthetic Omega-3 fatty acids.

35. The method of claim 29, 30, 31 or 32 wherein said one or more endothelial cell anti-inflammatory agents are selected from the group consisting of eicosapentaenoic acid, docosahexaenoic acid, alpha-linolenic acid, derivatives of eicosapentaenoic acid, derivatives of docosahexaenoic acid, derivatives of alpha-linolenic acid and fatty acid compound derivatives.

36. The method of claim 29, 30, 31 or 32 wherein said one or more endothelial cell anti-inflammatory agents are selected from the group consisting of eicosapentaenoic acid, docosahexaenoic acid, alpha-linolenic acid, derivatives of eicosapentaenoic acid, derivatives of docosahexaenoic acid, derivatives of alpha-linolenic acid, phospholipids esters of linolenic acid, ethers of linolenic acid, sterol derivatives of linolenic acid and fatty acid compounds.

37. The method of claim 29, 30, 31 or 32 wherein said one or more endothelial cell anti-inflammatory agents are selected from the group consisting of eicosapentaenoic acid, docosahexaenoic acid, alpha-linolenic acid, derivatives of eicosapentaenoic acid, derivatives of docosahexaenoic acid, derivatives of alpha-linolenic acid, phospholipids esters of linolenic acid, ethers of linolenic acid, sterol derivatives of linolenic acid, phosphatidal choline esters of linolenic acid, phosphatidal ether of linolenic acid and sipolsterol ester of linolenic acid.

38. The method of claim 29, 30, 31 or 32 wherein said one or more nitric oxide generation promoting agents are selected from the group consisting of folic acid, folate, precursors of folic acid, precursors of folate, derivatives of folic acid, derivatives of folate, metabolites of folic acid, metabolites of folate and natural isomers of folate.

39. The method of claim 29, 30, 31 or 32 wherein said one or more nitric oxide generation promoting agents are selected from the group consisting of folic acid, folate, precursors of folic acid, precursors of folate, derivatives of folic acid, derivatives of folate, metabolites of folic acid, metabolites of folate, (6S)-tetrahydrofolic acid, derivatives of (6S)-tetrahydrofolic acid, 5-methyl-(6S)-tetrahydrofolic acid, derivatives of 5-methyl-(6S)-tetrahydrofolic acid, 5-formyl-(6S)-tetrahydrofolic acid, derivatives of 5-formyl-(6S)-tetrahydrofolic acid, 10-formyl-(6R)-tetrahydrofolic acid, derivatives of 10-formyl-(6R)-tetrahydrofolic acid, 5,10-methylene-(6R)-tetrahydrofolic acid, derivatives of 5,10-methylene-(6R)-tetrahydrofolic acid, 5,10-methenyl-(6R)-tetrahydrofolic acid, derivatives of 5,10-methenyl-(6R)-tetrahydrofolic acid, 5-formimino-(6S)-tetrahydrofolic acid, derivatives of 5-formimino-(6S)-tetrahydrofolic acid, 10-formyl-(6RS)-tetrahydrofolic acid, derivatives of 10-formyl-(6RS)-tetrahydrofolic acid, 5,10-methylene-(6RS)-tetrahydrofolic acid, derivatives of 5,10-methylene-(6RS)-tetrahydrofolic acid, 5,10-methenyl-(6RS)-tetrahydrofolic acid, derivatives of 5,10-methenyl-(6RS)-tetrahydrofolic acid, polyglutamyl and derivatives of polyglutamyl.

40. The method of claim 29, 30, 31 or 32 wherein said one or more antioxidant agents are selected from the group consisting of ascorbic acid, natural alpha-tocopherol, synthetic alpha-tocopherol, trimethyl tocopheryl acetate, alpha-tocopherol succinate, Vitamin A, flavonoids, caretenoids, alpha-lipoic acid, phenolic compounds and CoQ 10.

41. The method of claim 29, 30, 31 or 32 wherein said one or more antioxidant agents are selected from the group consisting of ascorbic acid, alpha-tocopherol, trimethyl tocopheryl acetate, alpha-tocopherol succinate, Vitamin A, flavonoids, beta-carotene, lutein, violaxanthin, neoxanthin, cryptoxanthin, phytofluene, phytoene, lycopene,neurosporene, lactucaxanthin, anhydrolutein, zeaxanthin, alpha-lipoic acid, oligomeric proanthocyanidins, anthocyanosides and CoQ 10.

42. The method of claim 29, 30, 31 or 32 wherein said one or more antioxidant agents are selected from the group consisting of natural alpha-tocopherol, synthetic alpha-tocopherol, beta-tocopherol, gamma-tocopherol, trimethyl tocopheryl acetate, tocopherol succinate, derivatives of alpha-tocopherol, derivatives of beta-tocopherol, derivatives of gamma-tocopherol, derivatives of trimethyl tocopheryl acetate, derivatives of tocopherol succinate, precursors of alpha-tocopherol, precursors of beta-tocopherol, precursors or gamma-tocopherol, precursors of trimethyl tocopheryl acetate, precursors of tocopherol succinate, metabolites of alpha-tocopherol, metabolites of beta-tocopherol, metabolites of gamma-tocopherol metabolites of trimethyl tocopheryl acetate, metabolites of tocopherol succinate, isomers of alpha-tocopherol, isomers of beta-tocopherol, isomers of gamma-tocopherol, isomers of trimethyl tocopheryl acetate, isomers of tocopherol succinate, derivatives of tocol, derivatives of tocotrienol, agents having Vitamin E functionality, Vitamin A, Vitamin C, flavonoids, beta-carotene, lutein, violaxanthin, neoxanthin, cryptoxanthin, phytofluene, phytoene, lycopene,neurosporene, lactucaxanthin, anhydrolutein, zeaxanthin, alpha-lipoic acid, oligomeric proanthocyanidins, anthocyanosides and CoQ 10.

43. The method of claim 29, 30, 31 or 32 wherein said one or more platelet aggregation lowering agents are selected from the group consisting of Vitamin B₆ and agents having Vitamin B₆ functionality.

44. The method of claim 29, 30, 31 or 32 wherein said one or more platelet aggregation lowering agents are selected from the group consisting of pyridoxine, pyridoxal, pyridoxamine, derivatives of pyridoxine, derivatives of pyridoxal, derivatives of pyridoxamine, precursors of pyridoxine, precursors of pyridoxal, precursors of pyridoxamine, metabolites of pyridoxine, metabolites of pyridoxal, metabolites of pyridoxamine, isomers of pyridoxine, isomers of pyridoxal, isomers of pyridoxamine and agents having Vitamin B₆ functionality.

45. The method of claim 29, 30, 31, 32, 34, 35, 36 or 37 wherein said one or more endothelial cell anti-inflammatory agents are present in an amount of about 650 mg or greater.

46. The method of claim 29, 30, 31, 32, 34, 35, 36 or 37 wherein said one or more endothelial cell anti-inflammatory agents are present in an amount of about 1.38 g or greater.

47. The method of claim 29, 30, 31, 32, 34, 35, 36 or 37 wherein said one or more endothelial cell anti-inflammatory agents are present in an amount of about 2.0 g or greater.

48. The method of claim 29, 30, 31, 32, 34, 35, 36 or 37 wherein said one or more endothelial cell anti-inflammatory agents are present in an amount of about 2.68 g or greater.

49. The method of claim 29, 30, 31, 32, 38 or 39 wherein said one or more nitric oxide generation promoting agents are present in an amount of about 0.4 mg to about 5 mg.

50. The method of claim 29, 30, 31, 32, 38 or 39 wherein said one or more nitric oxide generation promoting agents are present in an amount of about 0.8 mg to about 10 mg.

51. The method of claim 29, 30, 31, 32, 38 or 39 wherein said one or more nitric oxide generation promoting agents are present in an amount of about 1.2 mg to about 15 mg.

52. The method of claim 29, 30, 31, 32, 38 or 39 wherein said one or more nitric oxide generation promoting agents are present in an amount of about 1.6 mg to about 20 mg.

53. The method of claim 29, 30, 31, 32, 38 or 39 wherein said one or more nitric oxide generation promoting agents are present in an amount of about 2.0 mg to about 25 mg.

54. The method of claim 29, 30, 31, 32, 40, 41 or 42 wherein said one or more antioxidant agents are present with about 100 IU to about 2000 IU as Vitamin E.

55. The method of claim 29, 30, 31, 32, 40, 41 or 42 wherein said one or more antioxidant agents are present with about 100 mg to about 2000 mg as Vitamin C.

56. The method of claim 29, 30, 31, 32, 43 or 44 wherein said one or more platelet aggregation lowering agents are present with about 12.5 mg to about 500 mg as Vitamin B₆, an agent having Vitamin B₆ functionality or a combination thereof.

57. A composition comprising:
at least 250 mg of at least one Omega-3 fatty acid, Omega-3 fatty acid derivative or a combination thereof;
at least about 0.4 mg of folic acid, folate, folic acid derivative, folic acid metabolite, folate derivative, folate metabolite or a combination thereof;
at least about 10 IU or an equivalent mg amount of at least one antioxidant; and
at least about 12.5 mg of vitamin B₆, an agent having Vitamin B₆ functionality or a combination thereof, formulated in one or more dosage forms to be administered using consistent administration or intermittent administration .

58. The composition of claim 57 wherein said product is formulated as 2 to 20 dosage forms administered using consistent administration, intermittent administration or uneven administration.

59. The composition of claim 57 wherein said dosage forms include at least one gel capsule.

60. The composition of claim 57 wherein said at least one Omega-3 fatty acid, Omega-3 fatty acid derivative or a combination thereof, are selected from the group consisting of eicosapentaenoic acid, docosahexaenoic acid, alpha-linolenic acid, derivatives of eicosapentaenoic acid, derivatives of docosahexaenoic acid and derivatives of alpha-linolenic acid and further comprising less than 150 mg Omega-6 fatty acids.

61. The composition of claim 57 wherein said at least one Omega-3 fatty acid, Omega-3 fatty acid derivative or a combination thereof is a combination of eicosapentaenoic acid and docosahexaenoic acid having an eicosapentaenoic acid:docosahexaenoic acid weight ratio of 2.5:1 or greater.

62. The composition of claim 57 wherein said antioxidant is selected from the group consisting of Vitamin E, Vitamin C, Vitamin A, CoQ 10, beta-carotene and combinations thereof.

63. A composition comprising:
about 250 mg to about 1500 mg of at least one Omega-3 fatty acid, Omega-3 fatty acid derivative or a combination thereof;
about 0.4 to about 5 mg of folic acid, folate, folic acid derivative, folate derivative, folic acid metabolite, folate metabolite or a combination thereof;
about 10 IU to about 400 IU or equivalent mg amount of at least one antioxidant; and
about 12.5 to about 100 mg of Vitamin B₆, an agent having Vitamin B₆ functionality or a combination thereof.

64. The composition of claim 57 or 63 wherein said at least one Omega-3 fatty acid, Omega-3 fatty acid derivative or a combination thereof is present in a ratio of eicosapentaenoic acid:docosahexaenoic acid selected from the group consisting of 100:0, 90-100:0-10, 70-90:10-30, 50-70:30-50, 30-50:50-70,10-30:70-90 and 0-10:90-100.

65. A composition comprising:
at least one Omega-3 fatty acid, Omega-3 fatty acid derivative or a combination thereof in an amount of about 500 mg to about 3000 mg;
at least one folic acid, folate, folic acid derivative, folate derivative, folic acid metabolite or folate metabolite in an amount of about 0.8 to about 10 mg;
at least one antioxidant present in an amount of about 20 IU to about 800 IU; and
Vitamin B₆, an agent having Vitamin B₆functionality or a combination thereof, present in an amount of about 25 mg to about 200 mg.

66. A composition comprising:
about 750 mg to about 4500 mg of one or more Omega-3 fatty acids selected from the group consisting of eicosapentaenoic acid, docosahexaenoic acid, alpha-linolenic acid and combinations thereof;
about 1.2 mg to about 15 mg of folic acid, folate, folic acid derivative, folate derivative, folic acid metabolite, folate metabolite or a combination thereof;
about 30 IU to about 1200 IU of one or more antioxidants; and
about 37.5 mg to about 300 mg of Vitamin B₆, an agent having Vitamin B₆ functionality or a combination thereof.

67. A composition comprising:
about 1000 mg to about 6000 mg of an Omega-3 fatty acid selected from the group consisting of eicosapentaenoic acid, docosahexaenoic acid, alpha-linolenic acid and combinations thereof;
about 1.6 mg to about 20 mg folic acid, folate, folic acid derivative, folate derivative, folic acid metabolite, folate metabolite or combinations thereof;
about 40 IU to about 1600 IU of one or more antioxidants; and
about 50 mg to about 400 mg Vitamin B₆, an agent having Vitamin B₆ functionality or a combination thereof.

68. A composition comprising:
about 1250 mg to about 7500 mg of an Omega-3 fatty acid selected from the group consisting of eicosapentaenoic acid, docosahexaenoic acid, alpha-linolenic acid and combinations thereof;
about 2 mg to about 25 mg of folic acid, folate, folic acid derivative, folate derivative, folic acid metabolite, folate metabolite and combinations thereof;
about 50 IU to about 2000 IU of one or more antioxidants; and
about 67.5 mg to about 500 mg vitamin B₆, an agent having Vitamin B₆ functionality or a combination thereof.

69. The composition of claim 57, 63, 65, 66, 67 or 68 wherein said composition is formulated in dosage forms comprising one or more gel capsules containing at least one Omega-3 fatty acid, Omega-3 fatty acid derivative or a combination thereof and some portion of said antioxidant.

70. The composition of claim 57, 63, 65, 66, 67 or 68 further comprising one or more agents selected from the group consisting of HMG CoA reductase inhibitors, anti-inflammatory agents, statin drugs, antiplatelet drugs, homocysteine lowering drugs, fibrates, fibric acid, gemfibrozil, fenofibrate and derivatives thereof.

71. A composition for promoting or maintaining cardiovascular health comprising:
about 1000 mg of one or more Omega-3 fatty acids, about 300 IU of Vitamin E and about 70 mg Omega-6 fatty acids in two or more gel capsules; and
about 4.0 mg of folic acid, about 37.5 mg of Vitamin B₆, about 50 mg Vitamin C, about 1000 mg calcium, and about 800 IU Vitamin D in two or more tablets, caplets or capsules for administration to promote or maintain cardiovascular health.

72. The composition of claim 1, 2, 3 or 4 further comprising artificial sweeteners, aromatics, flavoring agents and combinations thereof.

73. A method for prevention, stabilization, reversal or treatment of coronary artery disease or cerebrovascular disease comprising:
determining cardiovascular risk level of a human prior to administering a composition comprising an effective amount of at least one Omega-3 fatty acid or Omega-3 fatty acid derivative, an effective amount of folic acid or folate, an effective amount of one or more antioxidants and an effective amount of Vitamin B₆, an agent having Vitamin B₆ functionality or a combination thereof in a dosage formulation effective for said cardiovascular risk level.

74. The method of claim 73 wherein said daily dosage formulation is increased upon said human moving to a higher cardiovascular risk level.

75. The method of claim 73 wherein a human with a very low cardiovascular risk level is administered a dosage formulation comprising:
at least about 250 mg of at least one Omega-3 fatty acid selected from the group consisting of eicosapentaenoic acid, docosahexaenoic acid, alpha-linolenic acid and combinations thereof;
at least about 0.4 mg of folic acid or folate;
at least about 10 IU of antioxidant; and
at least about 12.5 mg of vitamin B₆, an agent having Vitamin B₆functionality or a combination thereof.

76. The method of claim 73 wherein a human with a low cardiovascular risk level is administered a dosage formulation comprising:
at least about 500 mg of at least one Omega-3 fatty acid selected from the group consisting of eicosapentaenoic acid, docosahexaenoic acid, alpha-linolenic acid and combinations thereof;
at least about 0.8 mg of folic acid or folate;
at least about 20 IU of antioxidant; and
at least about 25 mg of Vitamin B₆ an agent having Vitamin B₆ functionality or a combination thereof.

77. The method of claim 73 wherein a human with a moderate cardiovascular risk level is administered a dosage formulation comprising:
at least about 750 mg of at least one Omega-3 fatty acid selected from the group consisting of eicosapentaenoic acid, docosahexaenoic acid, alpha-linolenic acid and combinations thereof;
at least about 1.2 mg of folic acid or folate;
at least about 30 IU of antioxidant; and
at least about 37.5 mg of Vitamin B₆, an agent having Vitamin B₆functionality or a combination thereof.

78. The method of claim 73 wherein a human with a high cardiovascular risk level is administered a dosage formulation comprising:
at least about 1.0 g of at least one Omega-3 fatty acid selected from the group consisting of eicosapentaenoic acid, docosahexaenoic acid, alpha-linolenic acid and combinations thereof;
at least about 1.6 mg of folic acid or folate;
at least about 40 IU of antioxidant; and
at least about 50 mg of Vitamin B₆, an agent having Vitamin B₆functionality or a combination thereof.

79. The method of claim 73 wherein a patient with a very high cardiovascular risk level is administered a dosage formulation comprising:
at least about 1250 mg of at least one Omega-3 fatty acid selected from the group consisting of eicosapentaenoic acid, docosahexaenoic acid, alpha-linolenic acid and combinations thereof;
at least about 2 mg of folic acid or folate;
at least about 50 IU of antioxidant; and
at least about 67.5 mg of Vitamin B₆, an agent having Vitamin B₆functionality or a combination thereof.

80. A composition for prevention, stabilization, reversal or treatment of coronary artery disease and cerebrovascular disease comprising:
at least 250 mg of at least one Omega-3 fatty acid or Omega-3 fatty acid derivative;
at least about 0.4 mg of folic acid, folate, folic acid derivative, folate derivative, folic acid metabolite, folate metabolite or a combination thereof;
at least about 10 IU or equivalent mg amount of at least one antioxidant; and
at least about 12.5 mg of Vitamin B₆, an agent having Vitamin B₆functionality or a combination thereof, in one or more dosage forms for administration to humans of a very low cardiovascular risk level.

81. The composition of claim 80 wherein said composition is doubled for administration to humans of a low cardiovascular risk level.

82. The composition of claim 80 wherein said composition is tripled for administration to humans of a moderate cardiovascular risk level.

83. The composition of claim 80 wherein said composition is quadrupled for administration to humans of a high cardiovascular risk level.

84. The composition of claim 80 wherein said composition is quintupled for administration to humans of a very high cardiovascular risk level.

85. The composition of claim 80 wherein said composition is increased based on a human's cardiovascular risk level.

86. The composition of claim 1, wherein said composition is useful in the prevention, stabilization, reversal or treatment of inflammatory diseases selected from the group consisting of atherosclerosis, cirrhosis, mucositis, chronic pancreatitis, inflammatory bowel disease and asthma.

87. The composition of claim 1 wherein said composition is useful in the prevention, stabilization, reversal or treatment of an autoimmune disease, a misfolding disease, a degenerative disease, a neurological disease, topical conditions, a disease affecting behavior, reproductive conditions, diseases associated with inflammation, and ophthalmic diseases.

88. The composition of claim 1 wherein said composition is useful in the prevention, stabilization, reversal or treatment of cystic fibrosis, rheumatoid arthritis, glomerulosclerosis, pulmonary fibrosis or systemic lupus erythematosis.

89. A method of packaging a composition comprising:
packaging said composition in a container having indicia indicating dosage level dependent upon cardiovascular risk level of human.

90. A method of administering a composition comprising:
administering a cardiovascular health promoting or maintaining composition dosage to a human or other animal and increasing said dosage or decreasing said dosage based on cardiovascular risk level of said human or other animal.

91. A method of administering a composition comprising:
administering in one or more dosage forms about 250 mg to about 1500 mg of one or more endothelial cell anti-inflammatory agents to a human or other animal of very low cardiovascular risk;
administering in one or more dosage forms about 500 mg to about 3000 mg of one or more endothelial cell anti-inflammatory agents to a human or other animal of low cardiovascular risk;
administering in one or more dosage forms about 750 mg to about 4500 mg of one or more endothelial cell anti-inflammatory agents to a human or other animal of moderate cardiovascular risk;
administering in one or more dosage forms about 1000 mg to about 6000 mg of one or more endothelial cell anti-inflammatory agents to a human or other animal of high cardiovascular risk; or
administering in one or more dosage forms about 1250 mg to about 7500 mg of one or more endothelial cell anti-inflammatory agents to a human or other animal of very high cardiovascular risk;
for prevention, stabilization, reversal or treatment of coronary artery disease or cerebrovascular disease.

92. A method of administering a composition comprising:
administering in one or more dosage forms about 0.4 mg to about 5 mg of one or more nitric oxide generation promoting agents to a human or other animal of very low cardiovascular risk;
administering in one or more dosage forms about 0.8 mg to about 10 mg of one or more nitric oxide generation promoting agents to a human or other animal of low cardiovascular risk;
administering in one or more dosage forms about 1.2 mg to about 15 mg of one or more nitric oxide generation promoting agents to a human or other animal of moderate cardiovascular risk;
administering in one or more dosage forms about 1.6 mg to about 20 mg of one or more nitric oxide generation promoting agents to a human or other animal of high cardiovascular risk; or
administering in one or more dosage forms about 2 mg to about 25 mg of one or more nitric oxide generation promoting agents to a human or other animal of very high cardiovascular risk;
for prevention, stabilization, reversal or treatment of coronary artery disease or cerebrovascular disease.

93. A method of administering a composition comprising:
administering in one or more dosage forms about 10 IU to about 400 IU of one or more antioxidant agents to a human or other animal of very low cardiovascular risk;
administering in one or more dosage forms about 20 IU to about 800 IU of one or more antioxidant agents to a human or other animal of low cardiovascular risk;
administering in one or more dosage forms about 30 IU to about 1200 IU of one or more antioxidant agents to a human or other animal of moderate cardiovascular risk;
administering in one or more dosage forms about 40 IU to about 1600 IU of one or more antioxidant agents to a human or other animal of high cardiovascular risk; or
administering in one or more dosage forms about 50 IU to about 2000 IU of one or more antioxidant agents to a human or other animal of very high cardiovascular risk;
for prevention, stabilization, reversal or treatment of coronary artery disease or cerebrovascular disease.

94. A method of administering a composition comprising:
administering in one or more dosage forms about 12.5 mg to about 100 mg of one or more platelet aggregation lowering agents to a human or other animal of very low cardiovascular risk;
administering in one or more dosage forms about 25 mg to about 200 mg of one or more platelet aggregation lowering agents to a human or other animal of low cardiovascular risk;
administering in one or more dosage forms about 37.5 mg to about 300 mg of one or more platelet aggregation lowering agents to a human or other animal of moderate cardiovascular risk;
administering in one or more dosage forms about 50 mg to about 400 mg of one or more platelet aggregation lowering agents to a human or other animal of high cardiovascular risk; or
administering in one or more dosage forms about 67.5 mg to about 500 mg of one or more platelet aggregation lowering agents to a human or other animal of very high cardiovascular risk;
for prevention, stabilization, reversal or treatment of coronary artery disease or cerebrovascular disease.

95. The composition of claim 1, 2, 3, 4, 57, 63, 65, 66, 67, 68, 71 or 80 wherein said composition is effective in reducing Omega-6 fatty acid to Omega-3 fatty acid intake ratio by about 5 percent to about 50 percent.

96. A method of administering a composition comprising:
administering in one or more dosage forms about 250 mg to about 1500 mg of one or more endothelial cell anti-inflammatory agents and one or more agents selected from the group consisting of nitric oxide generation promoting agents, antioxidant agents and platelet aggregation lowering agents, to a human or other animal of very low cardiovascular risk;
administering in one or more dosage forms about 500 mg to about 3000 mg of one or more endothelial cell anti-inflammatory agents and one or more agents selected from the group consisting of nitric oxide generation promoting agents, antioxidant agents and platelet aggregation lowering agents, to a human or other animal of low cardiovascular risk;
administering in one or more dosage forms about 750 mg to about 4500 mg of one or more endothelial cell anti-inflammatory agents and one or more agents selected from the group consisting of nitric oxide generation promoting agents, antioxidant agents and platelet aggregation lowering agents, to a human or other animal of moderate cardiovascular risk;
administering in one or more dosage forms about 1000 mg to about 6000 mg of one or more endothelial cell anti-inflammatory agents and one or more agents selected from the group consisting of nitric oxide generation promoting agents, antioxidant agents and platelet aggregation lowering agents, to a human or other animal of high cardiovascular risk; or
administering in one or more dosage forms about 1250 mg to about 7500 mg of one or more endothelial cell anti-inflammatory agents and one or more agents selected from the group consisting of nitric oxide generation promoting agents, antioxidant agents and platelet aggregation lowering agents, to a human or other animal of very high cardiovascular risk;
for prevention, stabilization, reversal or treatment of coronary artery disease or cerebrovascular disease.
